(19) United States
(12) Patent Application Publication
(10) Pub. No.: US 2019/0053833 A1
(43) Pub. Date: Feb. 21, 2019

(54) SPINAL MULTI-LEVEL FACET JOINT STABILIZATION SYSTEM

(71) Applicant: LinkSPINE, Inc., Columbia, MD (US)

(72) Inventor: Christopher D. CHAPUT, Temple, TX (US)

(73) Assignee: LinkSPINE, Inc., Columbia, MD (US)

(21) Appl. No.: 15/766,548

(22) PCT Filed: Oct. 10, 2016
(86) PCT No.: PCT/US2016/056242

§ 371 Date: Apr. 6, 2018

Related U.S. Application Data
(60) Provisional application No. 62/239,762, filed on Oct. 9, 2015.

(51) Int. Cl. A61B 17/70

(52) U.S. Cl. CPC A61B 17/7064; A61B 17/7037; A61B 17/7032

(57) ABSTRACT

A stabilization implant for a facet joint of a first vertebrae includes a support module and a transfacetal fastener, wherein the support module is provided on either side with a holder for the transfacetal fastener, the transfacetal fastener having a head and a main body traversing the facet joint. The transfacetal fastener may include a super-head to form a stacked double-head arrangement, a top carrier on a top side of the transfacetal fastener, and an upward extension element, which is pivotally attached to the top carrier and which reaches upward and has a fixation portion at its upper end, the fixation portion configured for an oblique fastening to cortical structure of a second, adjacent upper vertebrae.

### CROSS-REFERENCE TO RELATED APPLICATIONS

This application is a national stage application under 35 USC 371 of International Application No. PCT/US2016/056242, filed Oct. 10, 2016, which claims priority to U.S. Provisional Patent Application No. 62/239,762, filed Oct. 9, 2015, the contents of each of which are incorporated herein by reference in their entirety.

### FIELD OF INVENTION

The invention relates to an implant for stabilization of a facet joint of a vertebrae. It comprises a support module and a transfacetal fastening means. More specifically it relates to an upward extension for an oblique fastening to a cortical structure of an adjacent upper vertebrae, thereby giving multi-level stabilization.

### BACKGROUND OF THE INVENTION

In the human body, a central structural element of the human skeleton is the spine. It comprises a plurality of vertebrae which are arranged one above another for the transfer of loads and are connected to one another with articulation to allow movements. For transfer of load between the vertebrae, an intervertebral disk is arranged between the vertebral bodies of neighbouring vertebrae, filling up an interspace between the relatively flat cover surfaces of the vertebral bodies. For articulation, upper joint protrusions and lower joint protrusions are provided on either side of each vertebrae. They form an articulated connection termed facet joint between two adjacent vertebrae. Due to wear or disease, the articulated connection of two neighbouring vertebrae may be damaged. This may lead to a restricted movement, pain or even loss of mobility. Various approaches have become known for treatment. In particular, a definite improvement can be achieved by stabilizing the facet joint. In many fields, this is done by immobilizing the facet joint by a fixed connection, termed fusion of the facet joint.

For effecting of such a stabilization of the facet joint, a fusion implant is known which comprises two long bone screws each of which is screwed through both of the facets forming a facet joint (WO 2012/072733 A1). This known fusion implant offers the advantage of relatively simple implantability because it only has small dimensions and therefore can be implanted even in minimally invasive surgery. However, this known fusion implant requires a relatively strong and intact bone structure of the vertebral body.

Quite often, the adjacent structure is not strong enough. In such a case, further stabilization is required necessitating extensive stabilization systems. Their implantation may require massively invasive surgery. This is stressful for the patient. Further, quite often it cannot be determined in advance whether such stabilization would be indeed required. Intra-operative switchover to such a system adds to complexity and therefore risk of the whole surgery process.

### SUMMARY OF THE INVENTION

It is an objective of aspects of the invention to provide an improved implant which is more flexible in usage.

According to an aspect of the invention, a stabilization implant is provided for a facet joint of a first vertebrae comprising a support module and a transfacetal fastening means, wherein the support module is provided on either side with a holder for transfacetal fastening means, the transfacetal fastening means having a head and a main body traversing the joint, wherein the transfacetal fastening means further comprises a super-head to form a stacked double-head arrangement, a top carrier on a top side of the support module, and an upward extension element which is pivotally attached to the top carrier, reaches upward and has a fixation portion at its upper end, the fixation portion being configured for an oblique fastening to a cortical structure of a second, adjacent upper vertebrae.

The term “upward extension element” relates to an extension element that is reaching predominantly in an upward direction (as opposed to a horizontal direction), i.e. away from the transfacetal fastening means which are positioned lower.

An aspect of the invention is based on the idea to provide a combination of a facet joint immobilization implant with a stabilizing implant which is anchored to a different, upper vertebrae. This is effected by providing a double head in a stacked arrangement on the transfacetal fastening element, thereby giving an additional super-head. This super-head allows easy attachment of the parts required for effecting stabilization. If no such stabilization was required then the super-head could be left unused or the transfacetal fastening element (preferably a screw) is easily exchanged against a conventional one without super-head. By virtue of this, an intra-operative switchover from/to stabilization is extremely feasible without effort, thus giving more flexibility to the surgeon and reducing additional risky tasks. This flexibility is even enhanced by the top carrier comprising a jump bar, which is preferably dimensioned such as to bridge an intermediate vertebrae between said first and second vertebrae. The term “bridging” means that the jump bar is not fixated at the intermediate vertebra, i.e. it spans the intermediate vertebra in a cantilever manner (cantilevered). Thereby, a truly multi-level stabilization is achieved. Preferably, the upward extension element has a threaded portion configured for an oblique fastening to a cortical structure of the upper vertebrae. By affixing to the rather strong cortical structure, a solid fixation can be achieved. “Oblique” preferably means a diverging angle of 15° or more. The oblique angle of fastening provides two further advantages, namely increased mechanical strength due to a longer thread length and easier mounting from below due to the oblique angle.

Moreover, the stabilization system makes double use of the transfacetal fastening elements, thereby reducing the total number of fastening elements required. Conventionally, six bone screws were required for providing a multi-level stabilization, and the invention just needs four.

Preferably, the jump bar is shorter than a distance between the first and second vertebrae, preferably by at least half of a height of a vertebra.

Preferably, the support module is laterally expandable. This provides for additional adjustability and improved fitment to the vertebrae.

Preferably, the upward extension element is linked to the top carrier by a lockable polyaxial joint. Thereby, the orientation of the upward extension could be adjusted in a wide range in order to effect fixation at the upper vertebrae.

The lockable polyaxial joint preferably comprises a sleeve having a reduced width at its front. The sleeve forms a polyaxial seat for the upward extension element. The reduced width secures a head portion head of the upward extension in that seat. The range for said polyaxiality could be +50 degrees or more in upward direction. Further, it is preferred that the sleeve comprises a tension cage in its interior and a pressing element, wherein the tension cage is configured to tiltably engage the head portion and the pressing element is configured to squeeze the tension cage for arresting of the head portion. Under a pressing force exerted by the pressing element the tension cage which encloses the head portion is pressed against said head portion, thereby effecting a press-fit which ensures a stable angle fixation.

A range of motion for polyaxial movement of the upward extension element is preferably limited by a skirt surrounding the head portion. However, in order to provide a sufficient range of angular motion to the upward direction, the rim is preferably slanted in respect to a center axis of the sleeve. Thereby, the range of angular motion is the largest in an upward direction, at the expense of the range of motion in a downward direction which is of no interest in such a configuration. As a result, the slanted orientation of the rim gives a favourable bias for the angular range of motion of said polyaxial attachment of the upward fastening element.

In a preferred embodiment, the super-head is ball shaped and the head is preferably cylindrically or conically shaped. The ball shaping of the super-head allows a rather high degree of angular motion. Further, the ball shape is convenient for use and allows attachment of the top carrier in any position. Further, the ball shape is low in terms of danger of irritating surrounding tissue.

On another token, the super-head is polyaxially held in a second sleeve having a reduced width at its front. The second sleeve operates in a manner similar to the sleeve of the upward extension element, i.e. combining wide angular adjustability with strong fixation by a press fit. To this end, the second sleeve comprises a second tension cage in its interior and a second pressing element, wherein the second tension cage is configured to tiltably engage the super-head and the second pressing element is configured to squeeze the second tension cage to the super-head.

The sleeve and/or the second sleeve preferably comprise at least one slot in its rear portion, the slot being configured for reception of a jump bar, the jump bar being preferably arrested by the second pressing element. By fixating of such a jump bar creation of a multi-level stabilization which jumps an intermediate vertebrae and leaves it untouched is feasible.

The invention further relates to an arrangement of a sleeve and fixation element, like a screw. For further details reference to the foregoing explanation is made.

### BRIEF DESCRIPTION OF THE DRAWINGS

The invention will now be explained in reference to the enclosed drawing, which shows an advantageous sample embodiment. There are shown:

FIG. 1 is a perspective view of the implant with jump bar;

FIGS. 2a-c are rear, lateral and bottom view of the implant of FIG. 1;

FIGS. 3a-b are views of a sleeve with slanted rim in disassembled and assembled states;

FIG. 4 is a detail view of a slanted rim on the sleeve;

FIG. 5 is a detail of a transfacetal screw with stacked double head; and

FIG. 6 is a detail cross section of a sleeve and the stacked double head.

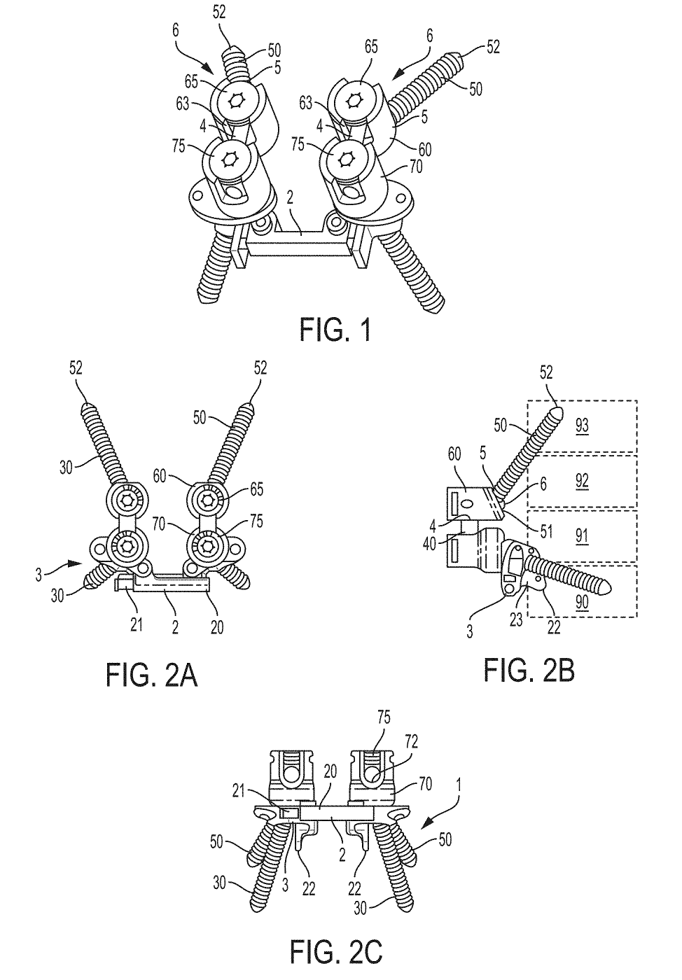

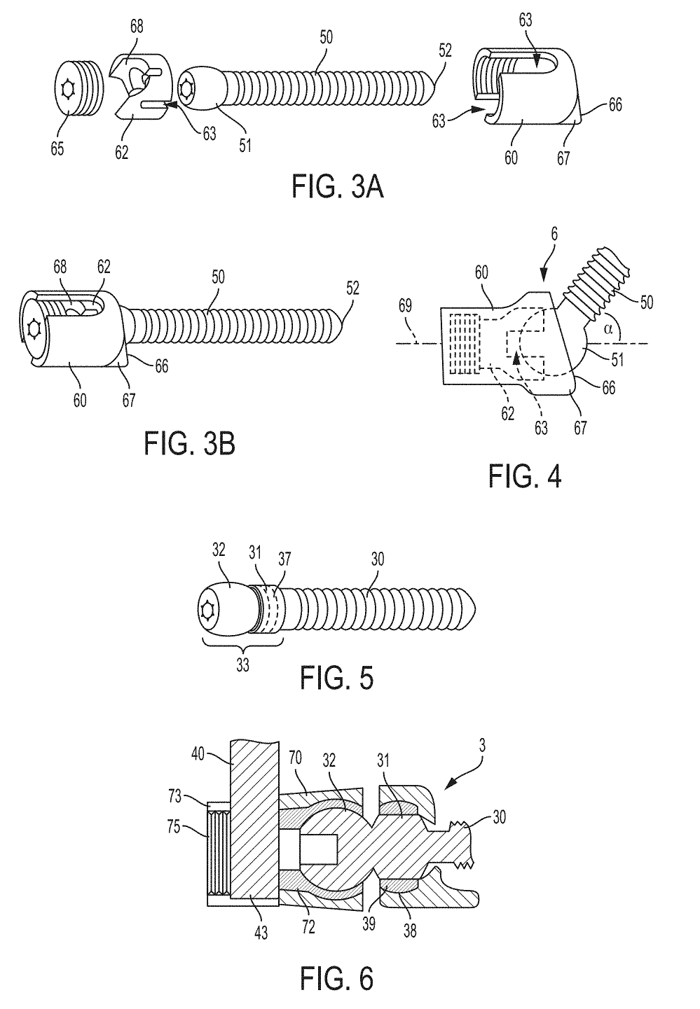

### DETAILED DESCRIPTION OF THE INVENTION

An embodiment of a stabilization implant 1 comprises a support module 2 and transfacetal fastening means 3. The support module 2 is designed in a bridge style. It comprises a rail body 20 in which a guide rod of a slider 21 is slidably mounted, so that the support module 2 can expand laterally. On a right end of the rail body 20 a holder 22 is arranged, and another holder 22 facing in opposite direction is arranged on a left end of the slider 21. Each holder 22 may have a jaw like surface 23 facing to the outside for interaction with a surface of a vertebrae 91.

The rail body 20 and the slider 21 cooperate in such a manner that by displacing the slider 21 a distance between the holders 22 changes, i.e. the support module 2 could be laterally expanded and vice versa. By adjusting the lateral expansion, the support module 2 may be configured such that it can bridge an interspace of different width in the lamina and to bring the holders 22 in a correct position for placing of screws as transfacetal fastening means 3 in order to join and immobilize the facet joints of said vertebrae 90.

The transfacetal fastening means 3 comprise a screw having a main body 30 with a thread and a head 31. The main body 30 is of sufficient length to traverse both facets of a facet joint of the two adjacent vertebrae 90 and 91, so that by tightening of the screw both facets are tightened against each other, thereby immobilizing the facet joint.

The head 31 of the transfacetal fastening means 3 is part of a stacked double-head arrangement 33 which features at its end an additional head superimposed on the head 31, this additional head being termed as super-head 32.

On the super head 32, an upward extension 5 is affixed by means of a top carrier 4 that comprises a jump bar 40. Said upward extension is linked to the top carrier by means of a lockable polyaxial joint 6. The upward extension is preferably a cortical bone screw having a threaded portion 50 which is configured for an oblique fastening to a cortical structure of an upper vertebrae 93. It features a pointed tip 52 configured for breaking the surface of upper vertebrae 93 in order to gain access for effecting stabilization. At the other end opposite to the tip 52, a ball shaped head 51 is provided.

The ball shaped head 51 is held in a sleeve 60 comprising a tensioning cage 62. Slots 63 are provided at a forward facing portion of the tension cage 62 being configured to engage the ball shaped head 51 in a pivotally manner. The tension cage 62 is at a forward position in the interior of the sleeve 60, and the ball shaped head 51 is moved into engagement by the tension cage 62 through an opening at the front end of the sleeve 60. From the back end of the sleeve 60, a pressing element 65 is to be mounted. To this end, the sleeve 60 is provided with an inner thread lining the nearly cylindrical wall of the interior of the sleeve 60. The pressing element 65 features a corresponding outer thread on its circumference which engages the inner thread. Thereby, the pressing element 65 moves forward by screwing it in and bears on the tension cage 62 which exerts a clamping force on the ball shaped head 51 of the cortical screw forming an upward extension 5. Thereby, the upward extension 5 is affixed in its angular position and provides additional stability to the support module 2.

In the depicted embodiment, the pressing element 65 does not bear directly on the tension cage 62, rather it does so via an intermediate piece which is an end portion of a jump bar 40. The jump bar 40 is attached with its other end portion 43 at the transfacetal fastening means 3. The length of the jump bar 40 is equivalent to the height of a vertebrae 92. As a result, an effect of inserting the jump bar is that the upward extension 5 is lifted upwards to such an extent that it engages the over next vertebrae 93, leaping one intermediate vertebrae 92. Thereby, a truly multi-level stabilization is achieved, wherein the stabilizing upward extension attaches to a vertebrae 93 which is two levels above that vertebrae 91 to which the support module 2 is attached.

For a proper attachment of the jump bar 40 to the sleeve 60, the latter is provided with two opposing slots 63 in a wall of the sleeve. The slots 63 are of such a width to allow a passage of an end portion of the jump bar 40. Thereby, the jump bar 40 passes transversely through the interior of the sleeve 60, between the pressing element 65 and the tension cage 62. In order to give a more rigid fixation, a rear face of the tension cage 62 features a concave portion forming a saddle 68. It is dimensioned such as to provide a fit to the jump bar 40. The pressure force exerted by the pressing element 65 is thus transmitted via the jump bar 40 to the tension cage 62. Thereby, both, the jump bar 40 as well as the tension cage 62, are receiving said pressure force and are locked in their respective positions.

On a front end of the sleeve 60 a circumferential skirt 67 is provided. It delimits with its rim 66 angular movement of the cortical screw of upward fixation element 5. The length of the skirt 67 is not uniform, rather it is shortest or zero toward a top position and longest toward a bottom position. As a result, the rim 66 is slanted against a center axis 69 of the sleeve 60. By virtue of this, the upward fixation element can reach a rather steep upward pointing position, up to an angle a of 50 degrees or even more, as opposed to a much limited movement in a downward direction.

For attachment of the top carrier 4 with its jump bar 40 to the super-head 32, a joint similar to the lockable polyaxial joint 6 is provided. It comprises a second sleeve 70 having opposing slots 73 in its wall, a second tension cage 72 engaging the ball shaped super-head 32 and a second pressing element 75, which exerts pressure force on the second tension cage 72 thereby securely fixing its angular position with respect to the ball shaped super-head 32. Further, between a rear face of the second tension cage 72 and the second pressing element 75 the lower portion 43 of the jump bar 40 is clamped, similar as described above in respect to the polyaxial locking joint 6.

Further, the head 31 is mounted my means of a tiltable spheroid ring 39 which is tiltably mounted in a complementary shaped seat 38 of the transfacetal fastening means 3. The head 31 may have a secondary thread 37 on its outer circumference which is configured to expand the spheroid ring 39 against its seat 38 in order to lock a desired angular position. Thereby, the angle for the screw with thread 30 could be adjusted for a secure immobilization of the facet joint between the vertebrae 90 and 91.

1. A stabilization implant for a facet joint of a first vertebra, the stabilization implant comprising a support module and a transfacetal fastener, wherein the support module comprises a holder for the transfacetal fastener, and the transfacetal fastener comprises a main body configured for traversing the facet joint of the first vertebra, a head and a super-head to that together form a stacked double-head arrangement, a top carrier located on a top side of the transfacetal fastener, and an upward extension element that is pivotally attached to the top carrier, extends upward, and has a fixation portion at an upper end, the fixation portion configured for oblique fastening to a cortical structure of a second vertebra.

2. The stabilization implant of claim 1, wherein the top carrier comprises a jump bar.

3. The stabilization implant of claim 1, wherein the upward extension element is linked to the top carrier by a lockable polyaxial joint.

4. The stabilization implant of claim 3, wherein the lockable polyaxial joint comprises a sleeve having a reduced width at a front, the width being smaller than a width of a head portion of the upward extension element.

5. The stabilization implant of claim 4, wherein the sleeve comprises a tension cage in an interior and a pressing element, wherein the tension cage is configured to tiltably engage the head portion and the pressing element is configured to squeeze the tension cage for arresting the head portion.

6. The stabilization implant of claim 5, wherein the sleeve has a skirt surrounding the head portion, wherein a rim of the skirt limits a tilting angle of the upward extension element.

7. The stabilization implant of claim 6, wherein the rim is slanted to a center axis of the sleeve, thereby allowing a greater tilt angle in an upward direction.

8. The stabilization implant of claim 4, wherein the sleeve comprises at least one slot in a rear portion, the at least one slot being configured for reception of a jump bar.

9. The stabilization implant of any of claim 1, wherein the upward extension element has a threaded portion configured for the oblique fastening to the cortical structure of the second vertebra.

10. The stabilization implant of claim 1, wherein the support module is laterally expandable.

11. The stabilization implant of claim 1, wherein the super-head is ball shaped.

12. The stabilization implant of claim 1, wherein the super-head is polyaxially held in a second sleeve having a reduced width at a front.

13. The stabilization implant of claim 12, wherein the second sleeve comprises a second tension cage in an interior and a second pressing element, wherein the second tension cage is configured to tiltably engage the super-head and the second pressing element is configured to squeeze the second tension cage to the super-head.

14. The stabilization implant of claim 12, wherein the second sleeve comprises at least one slot in a rear portion, the at least one slot being configured for reception of a jump bar.

15. A fixation assembly for an implant, the fixation assembly comprising a fixation element and a holder, wherein the fixation element comprises a ball head, and the holder comprises a sleeve, a tension cage, and a pressing element, wherein the pressing element is attached to an inner portion of the sleeve via an internal thread, the pressing element being configured to bear on the tension cage such that the tension cage is squeezed together for arresting an angular position of the ball head, wherein a surrounding skirt is provided on a front end of the sleeve, a rim of the skirt forming a limit for a range of angular motion of the fixation element, and wherein the rim is slanted with respect to a center axis of the sleeve.

16. The fixation assembly of claim 15, wherein the sleeve comprises at least one slot in a rear portion, the at least one slot being configured for reception of a jump bar.

17. The fixation assembly of claim 15, wherein the ball head is part of a double-head structure of the screw.

18. The fixation assembly of claim 17, wherein another head of the double-head structure is cylindrical or conical in shape and carries a secondary thread on an outer surface.

19. The fixation assembly of claim 18, wherein the fixation element is a screw and the secondary thread has a different pitch from a pitch of a thread of the screw.

20. The stabilization implant of claim 2, wherein the jump bar is dimensioned such as to bridge an intermediate vertebrae between the first and second vertebrae.

21. The stabilization implant of claim 20, wherein the jump bar is shorter than a distance between the first and second vertebrae.

22. The stabilization implant of claim 21, wherein the jump bar is shorter by at least half of a height of a vertebra.

23. The stabilization implant of claim 8, wherein the pressing element is configured for arresting the jump bar.

24. The stabilization implant of claim 11, wherein the head is conically shaped.

25. The stabilization implant of claim 14, wherein the second pressing element is configured for arresting the jump bar.

26. The fixation assembly of claim 15, wherein the fixation element is a screw.

27. The fixation assembly of claim 16, wherein the pressing element is configured for arresting the jump bar.
